(19) United States
(12) Patent Application Publication
(10) Pub. No.: US 2025/0195831 A1
(43) Pub. Date: Jun. 19, 2025

(54) CATHETER

(71) Applicant: ASAHI INTECC CO., LTD., Seto-shi (JP)

(72) Inventor: Patravut BOONSIRI, Seto-shi (JP)

(73) Assignee: ASAHI INTECC CO., LTD., Seto-shi (JP)

(21) Appl. No.: 19/066,965

(22) Filed: Feb. 28, 2025

Related U.S. Application Data
(63) Continuation of application No. PCT/JP2023/020434, filed on Jun. 1, 2023.

(30) Foreign Application Priority Data
Sep. 2, 2022 (JP) 2022-139741

(51) Int. Cl. A61M 25/00

(52) U.S. Cl. CPC A61M 25/0045; A61M 25/005

(57) ABSTRACT

Provided is a catheter including an inner layer 6a formed of a hollow tubular body having a lumen 3s, a braid 8 covering the inner layer 6a and including a first wire (9a to 9n) wound in a first direction and a second wire (7a to 7f, 5aa to 5bf) wound in a direction intersecting the first direction, and an outer layer 6b covering the braid 8. In particular, the second wire (7a to 7f, 5aa to 5bf) is constituted by a combination of a third wire (7a to 7f) having a width W1 of a predetermined length and a plurality of fourth wires (5aa to 5bf) each having a width W2 smaller than the width W1 of the third wire (7a to 7f).

### CROSS-REFERENCE TO RELATED APPLICATION

This application is a continuation application of PCT/JP2023/020434, which was filed on Jun. 1, 2023, and claims priority to Japanese Patent Application 2022-139741, which was filed on Sep. 2, 2022.

### TECHNICAL FIELD

The disclosure relates generally to a catheter used by being inserted into a lumen such as a blood vessel of a patient.

### BACKGROUND

Conventionally, a catheter used by being inserted into a lumen such as a blood vessel of a patient has been known. A doctor who performs a procedure inserts a guide wire into an inner cavity of the catheter, causes a distal end of the guide wire to protrude from a distal end of the catheter, causes the catheter to reach a lesion by leading the guide wire, and arranges a device such as a stent or an embolic coil at the lesion through the inner cavity of the catheter which has reached the lesion.

For example, Patent Literature 1 describes a catheter 1 for penetrating a constricted part including a flexible tube 2 having an inner cylinder 21, a braid 22 as a reinforcing layer arranged on an outer side of the inner cylinder 21, and an outer cylinder 23 covering the braid 22 (see FIGS. 1 and 2, etc.).

In addition, as for the braid 22 constituting the flexible tube 2, Patent Literature 1 describes that two kinds of a wire rod 22a and a wire rod 22b that are different in stiffness are wound around an outer side of the inner cylinder 21, thereby making it easier to adjust the strength and stiffness of a catheter main body (see paragraph [0035], etc.).

### CITATION LIST

### Patent Literature

Patent Literature 1: Japanese Unexamined Patent Application Publication No. 2015-195847

### SUMMARY

### BRIEF DESCRIPTION OF DRAWINGS

FIG. 1 is an overall view of a catheter according to a first embodiment of the present invention.

FIG. 2 is a longitudinal sectional view of a portion A in FIG. 1.

FIG. 3 is a perspective view illustrating a part of a braid constituting the catheter according to the first embodiment.

FIG. 4 is an enlarged view of a portion C in FIG. 3.

FIG. 5 is a view, corresponding to FIG. 4, of another braid constituting the catheter according to the first embodiment.

FIG. 6 is an enlarged view of a portion B in FIG. 2.

FIG. 7 is a view corresponding to FIG. 6 when the catheter according to the first embodiment is curved.

FIG. 8 is a view, corresponding to FIG. 6, of a braid constituting a catheter according to a second embodiment.

FIG. 9 is a view, corresponding to FIG. 6, of a braid constituting a catheter according to a third embodiment.

FIG. 10 is a view, corresponding to FIG. 6, of a braid constituting a catheter according to a fourth embodiment.

FIG. 11 is a view, corresponding to FIG. 6, of a braid constituting a catheter according to a fifth embodiment.

FIG. 12 is a view, corresponding to FIG. 6, of a braid constituting a catheter according to a sixth embodiment.

FIG. 13 is a view, corresponding to FIG. 6, of a braid constituting a catheter according to a seventh embodiment.

FIG. 14 is a view, corresponding to FIG. 6, of a braid constituting a catheter according to an eighth embodiment.

### EMBODIMENTS OF THE INVENTION

Hereinafter, embodiments of the present invention will be explained with reference to the drawings.

### First Embodiment

First, a first embodiment of the present invention will be explained. Note that drawings to be used in the present embodiment are exaggerated for ease of understanding, and dimensions thereof are different from the actual dimensions.

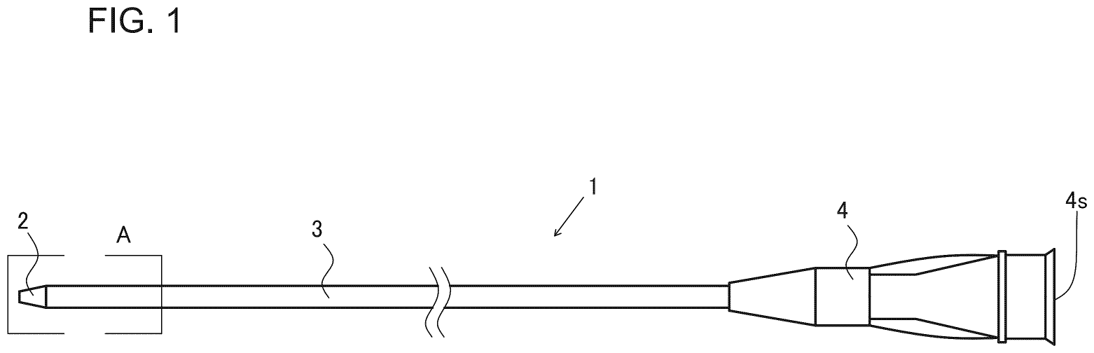

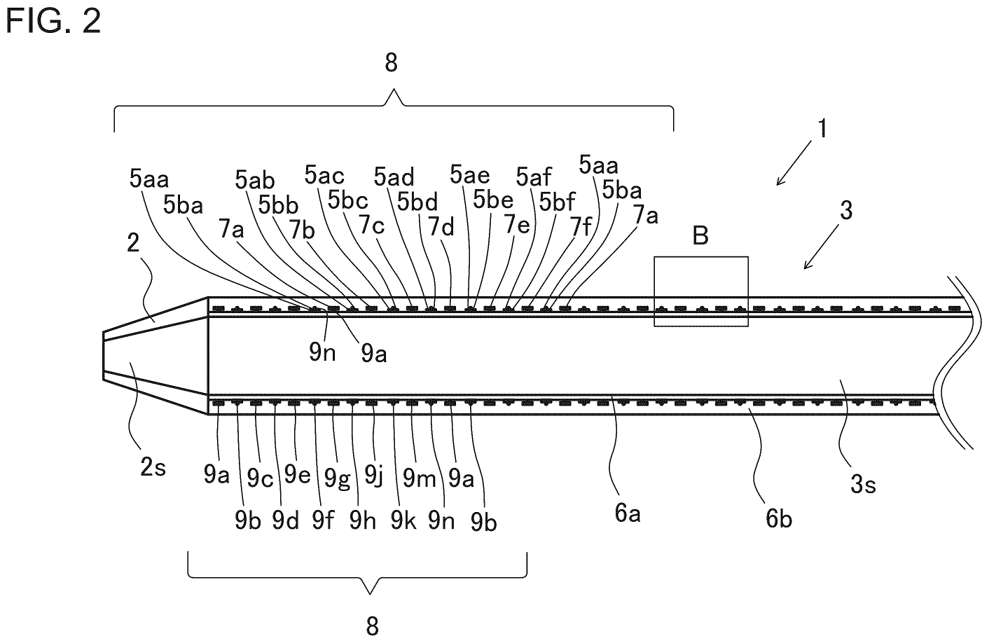

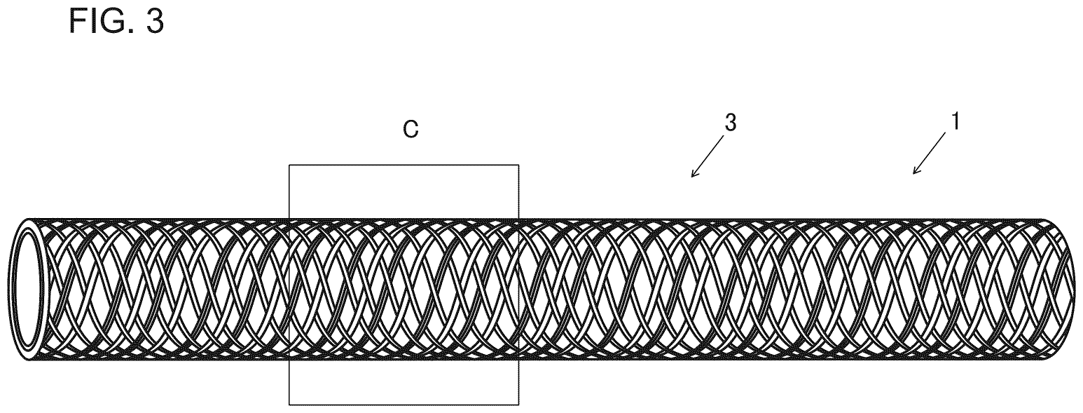

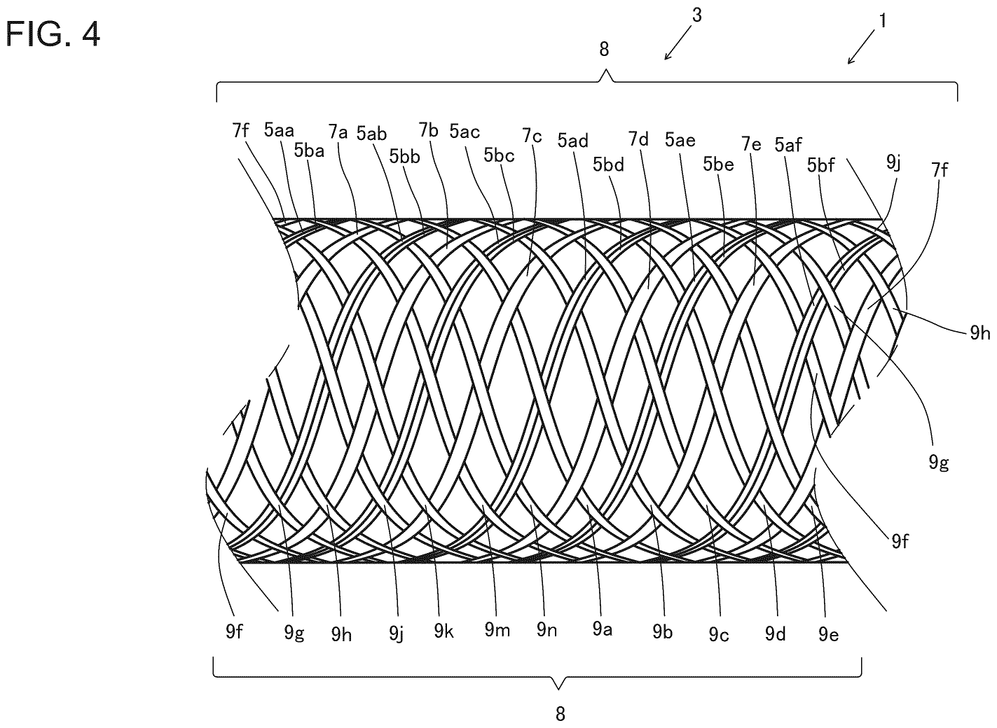

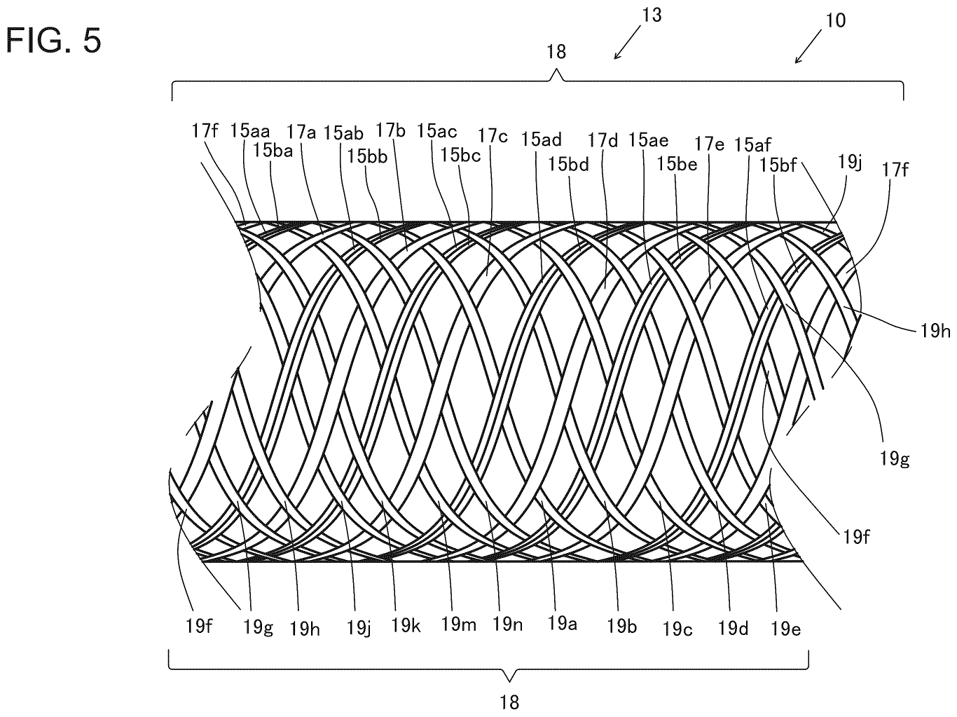

FIG. 1 is an overall view of a catheter according to a first embodiment of the present invention, FIG. 2 is a longitudinal sectional view of a portion A in FIG. 1, FIG. 3 is a perspective view illustrating a part of a braid constituting the catheter according to the first embodiment, FIG. 4 is an enlarged view of a portion C in FIG. 3, and FIG. 5 is a view, corresponding to FIG. 4, of another braid constituting the catheter according to the first embodiment.

As illustrated in FIG. 1, a catheter 1 according to the present embodiment includes a catheter main body 3, a distal tip 2 joined to a distal end of the catheter main body 3, and a connector 4 joined to a proximal end of the catheter main body 3.

As illustrated in FIG. 2, the catheter main body 3 includes an inner layer 6a having an inner cavity 3s, a braid 8 wound around an outer periphery of the inner layer 6a, and an outer layer 6b covering the inner layer 6a and the braid 8.

The inner layer 6a is a long hollow tubular body formed from a resin, and forms a lumen 3s for inserting a guide wire or other medical devices therein.

As the resin material forming the inner layer 6a, any material having biocompatibility may be used, and for example, polytetrafluoroethylene (PTFE), tetrafluoroethylene-perfluoroalkyl vinyl ether (PFA), copolymer tetrafluoroethylene-hexafluoropropylene copolymer (FEP), tetrafluoroethylene-ethylene copolymer (ETFE), polyvinylidene fluoride (PVDF), and the like can be used. In the present embodiment, polytetrafluoroethylene (PTFE) is used.

As illustrated in FIGS. 2 to 4, the braid 8 is formed by alternately weaving a first wire wound in a first direction with respect to the inner layer 6a and a second wire wound in a second direction intersecting with the first direction into a mesh shape.

To be more specific, the first wire of the present embodiment includes 12 first wires 9a, 9b, 9c, 9d, 9e, 9f, 9g, 9h, 9j, 9k, 9m, and 9n (hereinafter, referred to as “9a to 9n”) wound in the first direction with respect to the inner layer 6a on the outer periphery of the inner layer 6a.

On the other hand, the second wire includes six third wires 7a, 7b, 7c, 7d, 7e, and 7f (hereinafter, referred to as “7a to 7f”) and six sets of 12 fourth wires 5aa-5ba, 5ab-5bb, 5ac-5bc, 5ad-5bd, 5ae-5be, and 5af-5bf (hereinafter, referred to as “5aa to 5bf”), the third and fourth wires being wound in the second direction intersecting with the first direction on the outer periphery of the inner layer 6a.

Herein, for example, one set of “5aa-5ba” means that the fourth wire 5aa and the fourth wire 5ba form one set and are twisted with respect to the first wire (for example, 9a to 9n).

Therefore, the above-described six sets of “5aa-5ba”, “5ab-5bb”, “5ac-5bc”, “5ad-5bd”, “5ae-5be”, and “5af-5bf” mean that the respective two wires form one set and are twisted with respect to the first wire (for example, 9a to 9n).

The above-described first direction generally means, for example, a counterclockwise direction toward a distal end of the catheter 1, and the above-described second direction intersecting with the first direction means, for example, a clockwise direction toward the distal end of the catheter 1. However, the present embodiment and embodiments to be described below are not limited thereto, and the first direction and the second direction may be any directions as long as the first direction and the second direction intersect each other.

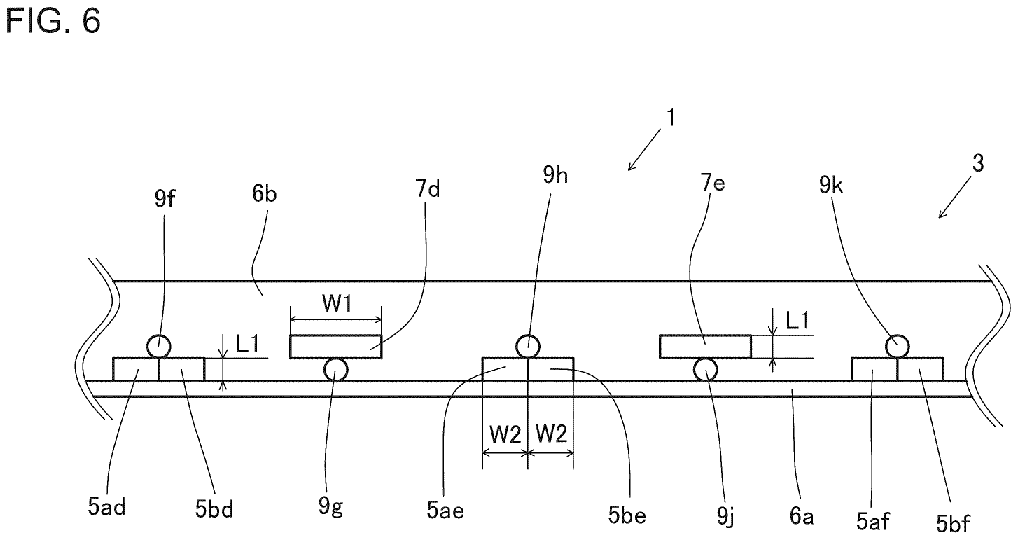

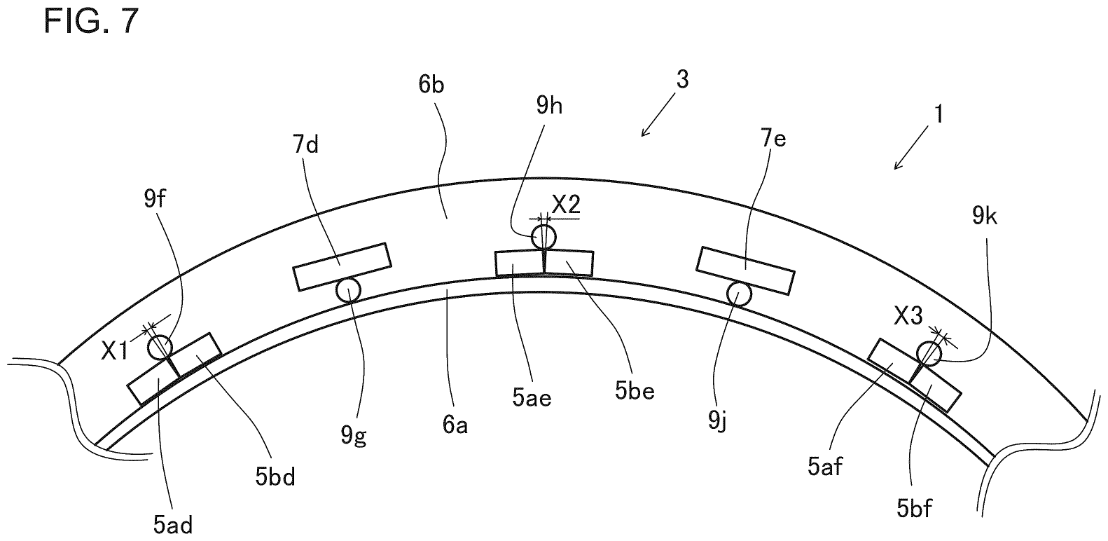

FIG. 6 is an enlarged view of a portion B in FIG. 2, and FIG. 7 is a view corresponding to FIG. 6 when the catheter of the first embodiment is curved.

As described above, the first wire of the present embodiment includes 12 first wires 9a to 9n wound in the first direction with respect to the inner layer 6a on the outer periphery of the inner layer 6a, and the second wire includes six third wires 7a to 7f and six sets of 12 fourth wires 5aa to 5bf, the third and fourth wires being wound in the second direction intersecting with the first direction on the outer periphery of the inner layer 6a.

As illustrated in FIG. 6, each of the first wires 9a to 9n in the present embodiment is a wire having a substantially circular transverse section, each of the third wires 7a to 7f constituting the second wire is a flat wire (a wire rod having a substantially rectangular transverse section) with a width=W1 and a thickness=L1, and each of the fourth wires 5aa to 5bf constituting the second wire is a flat wire (a wire rod having a substantially rectangular transverse section) with a width=W2 smaller than the width W1 of the third wires 7a to 7f and a thickness=L1.

In the present embodiment, the width of each of the fourth wires 5aa to 5bf is set to about ½ times the width of each of the third wires 7a to 7f.

Then, when the catheter main body 3 constituting the catheter 1 of the present embodiment is curved, as illustrated in FIG. 7, each space between the fourth wires 5aa and 5ba, between the fourth wires 5ab and 5bb, between the fourth wires 5ac and 5bc, between the fourth wires 5ad and 5bd, between the fourth wires 5ae and 5be, and between the fourth wires 5af and 5bf is slightly opened in a fan shape (in FIG. 7, a space between the fourth wires 5ad and 5bd=X1, a space between the fourth wires 5ae and 5be=X2, and a space between the fourth wires 5af and 5bf=X3), whereby the flexibility and the kink resistance of the catheter 1 can be improved.

In the present embodiment and embodiments to be described below, stiffness, flexibility, and kink resistance of the catheter can be adjusted by arrangement proportions of the third wires and the fourth wires.

For example, when the proportion of the third wires in the second wire is increased, the stiffness of the catheter can be further improved, and when the proportion of the fourth wires in the second wire is increased, the flexibility and kink resistance of the catheter can be further improved.

As materials of the twelve first wires 9a to 9n and the six third wires 7a to 7f and six sets of twelve fourth wires 5aa to 5bf, which constitute the braid 8, biocompatible metallic wires such as stainless steel, tungsten, and Ni—Ti alloys can be used. In the present embodiment, stainless steel is used for both wires.

As illustrated in FIG. 4, the braid 8 used in the present embodiment has a form in which the first wires and the second wires are alternately woven into a mesh shape with one wire or one set each, but the present invention is not limited to such a braid. For example, the braid 18 of a catheter main body 13 constituting a catheter 10 illustrated in FIG. 5, may be in a form in which 12 first wires (19a, 19b, 19c, 19d, 19e, 19f, 19g, 19h, 19j, 19k, 19m, and 19n) and six third wires (17a, 17b, 17c, 17d, 17e, 17f) and twelve fourth wires consisting of six sets of wire pairs (15aa-15ba, 15ab-15bb, 15ac-15bc, 15ad-15bd, 15ae-15be, and 15af-15bf) are alternately woven into a mesh shape with two wires each, or with one wire and one set each, and it can be also applied to a form in which the first wires and the second wires are alternately woven into a mesh shape with three or more wires each, two wires and one or more sets each, or one wire and two or more sets each.

Further, in the present embodiment, explanation has been made assuming that the number of the first wires is 12, the number of the second wires is six and six sets of two, but the present invention is not limited to these numbers, and the number of the first wires may be one or more, and the number of the second wires may be one and one set or more.

The outer layer 6b is formed of a resin and covers the inner layer 6a and the braid 8. A resin material forming the outer layer 6b is preferably a thermoplastic resin or a thermoplastic elastomer having biocompatibility. For example, a polyamide-based elastomer such as a polyether block amide copolymer, polyamide, polyimide, polyamide-imide, polyethylene terephthalate, polyethylene, polypropylene, polyurethane, ethylene, a vinyl acetate copolymer, polyvinyl chloride, or the like can be used, and a polyamide-based elastomer is used in the present embodiment.

The distal tip 2 joined to the distal end of the catheter main body 3 has a hollow cylindrical shape provided with a lumen 2s communicating with the lumen 3s of the catheter main body 3, and has an outer peripheral surface of a tapered shape tapered toward the most distal end portion.

As the resin material forming the distal tip 2, a polystyrene elastomer, polystyrene, a polyolefin elastomer, polyolefin, an ethylene-vinyl acetate copolymer, a polyvinyl chloride elastomer, polyvinyl chloride, a polyurethane elastomer, polyurethane, a polyester elastomer, polyester, a polyamide elastomer, polyamide, or the like having biocompatibility can be used. In the present embodiment, the polyurethane elastomer is used.

The connector 4 is formed of a resin, and includes a lumen 4s communicating with the lumen 3s of the catheter main body 3, and is joined to the proximal end of the catheter main body 3.

According to the catheter 1 of the present embodiment, there are included the inner layer 6a formed of the hollow tubular body having the lumen 3s, the braid 8 covering the inner layer 6a and constituted of the first wires 9a to 9n wound in the first direction and the second wires 7a to 7f and 5aa to 5bf wound in the direction intersecting the first direction, and the outer layer 6b covering the braid 8, and in particular, the second wires 7a to 7f and 5aa to 5bf are constituted by a combination of the third wires 7a to 7f having a width W1 of a predetermined length and the plurality of fourth wires 5aa to 5bf having a width W2 smaller than the width W1 of the third wires 7a to 7f, and therefore, the stiffness, flexibility, kink resistance of the catheter 1 can be easily adjusted by adjusting the combination of the third wires 7a to 7f and the plurality of fourth wires 5aa to 5bf.

In addition, according to the catheter 1 of the present embodiment, since the third wires 7a to 7f and the plurality of fourth wires 5aa to 5bf are alternately arranged, it is possible to easily manufacture the catheter 1 in which the stiffness, flexibility, and kink resistance are appropriately adjusted.

Further, according to the catheter 1 of the present embodiment, since each transverse section of the third wires 7a to 7f and the plurality of fourth wires 5aa to 5bf exhibits a substantially rectangular shape, a contact area with the inner layer 6a or the outer layer 6b can be increased without increasing an outer diameter of the catheter 1, and the kink resistance of the catheter 1 can be improved.

In addition, according to the catheter 1 of the present embodiment, since the transverse section of each of the first wires 9a to 9n exhibits a substantially circular shape, it is possible to increase the flexibility of the catheter 1 by reducing contact areas between the first wires 9a to 9n and the third wires 7a to 7f or the plurality of fourth wires 5aa to 5bf.

### Second Embodiment

Next, a second embodiment of the present invention will be explained. The drawings to be used in the present embodiment are also exaggerated for ease of understanding, and dimensions thereof are different from the actual dimensions.

Hereinafter, the second embodiment of the present invention will be explained, but since an overall view of a catheter is the same as FIG. 1, explanation thereof will be omitted, and portions common to the first embodiment will be denoted by the same reference numerals and explanation thereof will be omitted.

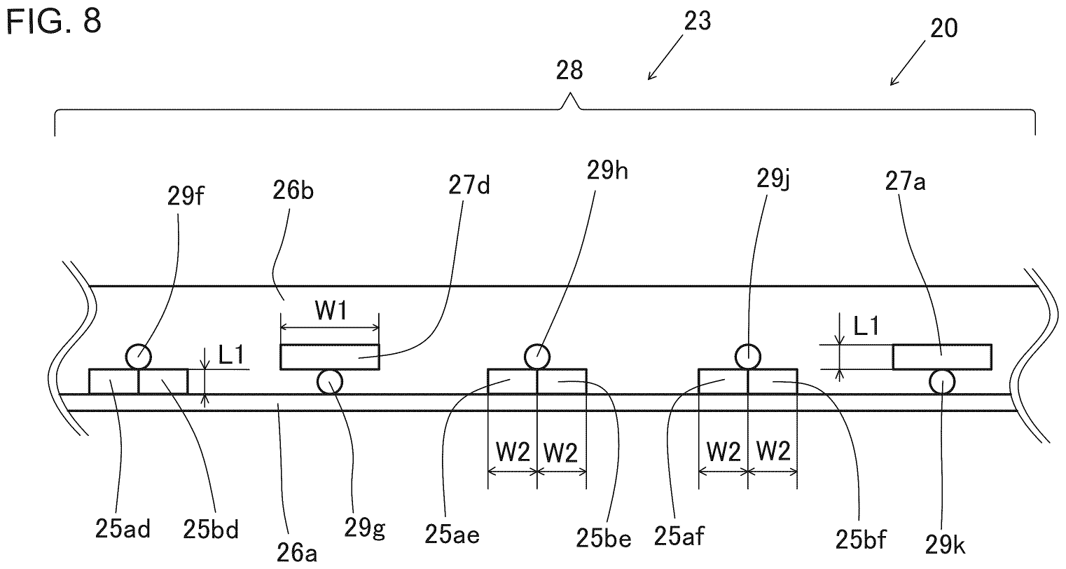

FIG. 8 is a view, corresponding to FIG. 6, of a braid constituting the catheter according to the second embodiment. A catheter 20 according to the second embodiment is different from the catheter 1 according to the first embodiment in braid configuration. Namely, in the braid 8 of the catheter 1, the third wire and the sets of fourth wires are alternately arranged, but in a braid 28 of the catheter 20, one third wire is arranged for every two sets of fourth wires.

Therefore, as described above, the catheter 20 according to the second embodiment can further improve the flexibility and kink resistance of the catheter as compared with the catheter 1 according to the first embodiment.

The catheter 20 according to the second embodiment includes a catheter main body 23, a distal tip 2 joined to a distal end of the catheter main body 23, and a connector 4 joined to a proximal end of the catheter main body 23, similar to the features shown in FIG. 1.

As illustrated in FIG. 8, the catheter main body 23 includes an inner layer 26a having an inner cavity 3s, the braid 28 wound around an outer periphery of the inner layer 26a, and an outer layer 26b covering the inner layer 26a and the braid 28.

The inner layer 26a is the same as the inner layer 6a of the first embodiment, and the outer layer 26b is also the same as the outer layer 6b of the first embodiment.

As illustrated in FIG. 8, the braid 28 is formed by weaving a first wire wound in a first direction with respect to the inner layer 26a and a second wire wound in a second direction intersecting with the first direction into a mesh shape.

To be specific, the first wire of the present embodiment includes 12 first wires (29a, 29b, 29c, 29d, 29e, 29f, 29g, 29h, 29j, 29k, 29m, and 29n (hereinafter, referred to as “29a to 29n”) wound in the first direction with respect to the inner layer 6a on the outer periphery of the inner layer 6a.

On the other hand, the second wire includes four third wires (27a, 27b, 27c, and 27d (hereinafter, referred to as “27a to 27d”)) and 16 fourth wires consisting of eight sets of wire pairs (25aa-25ba, 25ab-25bb, 25ac-25bc, 25ad-25bd, 25ae-25be, 25af-25bf, 25ag-25bg, and 25ah-25bh (hereinafter, referred to as “5aa to 5bh”)), the third and fourth wires being wound in the second direction intersecting with the first direction on the outer periphery of the inner layer 6a.

Although the first wires 29a to 29e and 29m to 29n, the third wires 27b to 27c, and the fourth wires 25aa-25ba, 25ab-25bb, 25ac-25bc, 25ag-25bg, and 25ah-25bh are not illustrated in FIG. 8, the first wires, the third wires, and the fourth wires of the present embodiment are arranged in order as illustrated in FIG. 8, similarly to the first wires, the third wires, and the fourth wires in FIG. 2.

Herein, for example, one set of “25aa-25ca” means that two fourth wires of the wire 25aa and the wire 25ba form one set and are twisted with respect to the first wire (for example, 29a to 29n).

Therefore, the above-mentioned eight sets of “25aa-25ca”, “25ab-25cb”, “25ac-25cc”, “25ad-25cd”, “25ae-25ce”, “25af-25cf”, “25ag-25cg”, “25ah-25ch” mean that the respective two wires form one set and are twisted with respect to the first wire (for example, 29a to 29n).

As illustrated in FIG. 8, each of the first wires 29a to 29n in the present embodiment is a wire having a substantially circular transverse section, each of the third wires 27a to 27d constituting the second wire is a flat wire (a wire rod having a substantially rectangular transverse section) with a width=W1 and a thickness=L1, and each of the fourth wires 25aa to 5bh constituting the second wire is a flat wire (a wire rod having a substantially rectangular transverse section) with a width=W2 smaller than the width W1 of the third wires 27a to 27d and a thickness=L1.

In the present embodiment, the width of each of the fourth wires 25aa to 25bh is set to about ½ times the width of each of the third wires 27a to 27d.

When the catheter main body 23 constituting the catheter 20 of the present embodiment is curved, each space between the fourth wires 25aa and 25ba, between the fourth wires 25ab and 25bb, between the fourth wires 25ac and 25bc, between the fourth wires 25ad and 25bd, between the fourth wires 25ae and 25be, between the fourth wires 5af and 5bf, between the fourth wires 25ag and 25bg, and between the fourth wires 5ah and 5bh is slightly opened in a fan shape as in the state illustrated in FIG. 7, whereby flexibility and kink resistance of the catheter can be improved.

As materials of the twelve first wires 29a to 29n and the four third wires 27a to 27d and eight sets of fourth wires 25aa to 25bh, which constitute the braid 28, biocompatible metallic wires such as stainless steel, tungsten, and Ni—Ti alloys can be used. In the present embodiment, stainless steel is used for both wires.

As illustrated in FIG. 4, the braid 28 used in the present embodiment has a form in which the first wires and the second wires are alternately woven into a mesh shape with one wire or one set each, but the present invention is not limited to such a braid, may be in a form in which the twelve first wires 29a to 29n and the four third wires (27a, 27b, 27c, 27d) and eight sets of fourth wires (25aa-25ba, 25ab-25bb, 25ac-25bc, 25ad-25bd, 25ae-25be, 25af-25bf, 25ag-25bg, and 25ah-25bh) are alternately woven into a mesh shape with two wires each, or with one wire and one set each, and it can be also applied to a form in which the first wires and the second wires are alternately woven into a mesh shape with three or more wires each, two wires and one or more sets each, or one wire and two or more sets each.

According to the catheter 20 of the present embodiment, there are included the inner layer 26a formed of a hollow tubular body having a lumen 3s, the braid 28 covering the inner layer 26a and constituted of the first wires 29a to 29n wound in the first direction and the second wires 27a to 27d and 25aa to 25bh wound in a direction intersecting the first direction, and the outer layer 26b covering the braid 28, and in particular, the second wires 27a to 27d and 25aa to 25bh are constituted by a combination of the third wires 27a to 27d having a width W1 of a predetermined length and the plurality of fourth wires 25aa to 25bh having a width W2 smaller than the width W1 of the third wires 27a to 27d, and therefore, the stiffness, flexibility, kink resistance of the catheter 20 can be easily adjusted by adjusting the combination of the third wires 27a to 27d and the plurality of fourth wires 25aa to 25bh.

In addition, according to the catheter 20 of the present embodiment, since each transverse section of the third wires 27a to 27d and the plurality of fourth wires 25aa to 25bh exhibits a substantially rectangular shape, a contact area with the inner layer 26a or the outer layer 26b can be increased without increasing an outer diameter of the catheter 20, and the kink resistance of the catheter 20 can be improved.

In addition, according to the catheter 20 of the present embodiment, since the transverse section of each of the first wires 29a to 29n exhibits a substantially circular shape, it is possible to increase the flexibility of the catheter 20 by reducing contact areas between the first wires 29a to 29n and the third wires 27a to 27d or the plurality of fourth wires 25aa to 25bh.

### Third Embodiment

Next, a third embodiment of the present invention will be explained. Drawings to be used in the present embodiment are also exaggerated for ease of understanding, and dimensions thereof are different from the actual dimensions.

Hereinafter, a third embodiment of the present invention will be explained, but since an overall view of a catheter is the same as FIG. 1, explanation thereof will be omitted, and portions common to the first embodiment will be denoted by the same reference numerals, and explanation thereof will be omitted.

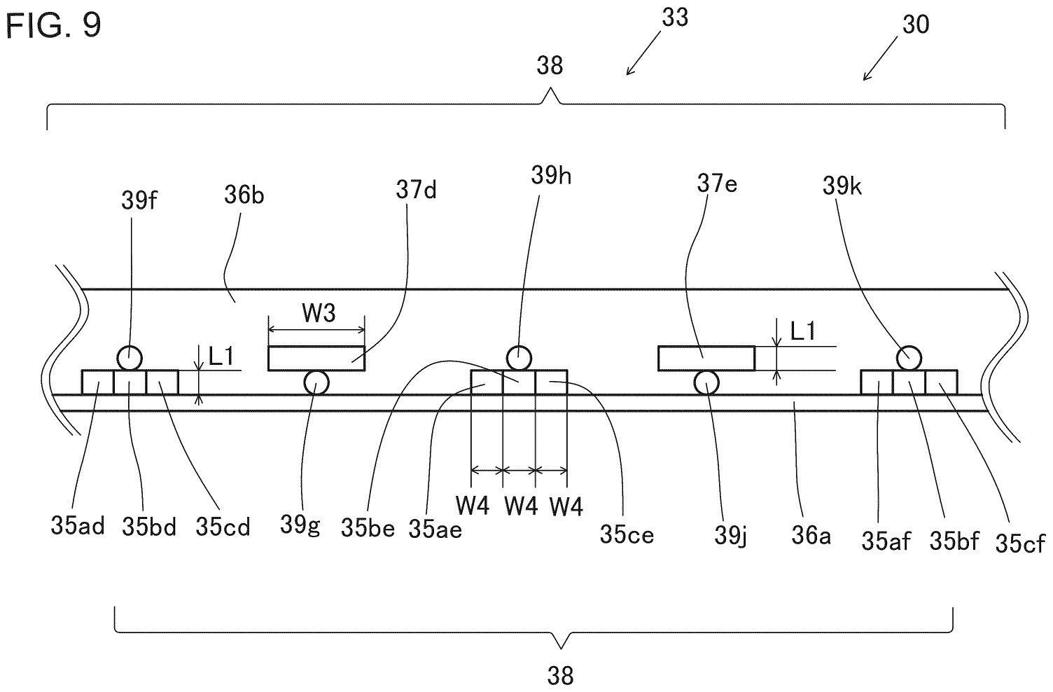

FIG. 9 is a view, corresponding to FIG. 6, of a braid constituting the catheter according to the third embodiment. A catheter 30 according to the third embodiment is different from the catheter 1 according to the first embodiment in the configuration of the fourth wire. Namely, in this embodiment a fourth wire constituting a second wire of the catheter 30 according to the present embodiment is constituted by three wires as one set.

The catheter 30 according to the present embodiment includes a catheter main body 33, a distal tip 2 joined to a distal end of the catheter main body 33, and a connector 4 joined to a proximal end of the catheter main body 33, similar to the features shown in FIG. 1.

As illustrated in FIG. 9, the catheter main body 33 includes an inner layer 36a having an inner cavity 3s, a braid 38 wound around an outer periphery of the inner layer 36a, and an outer layer 36b covering the inner layer 36a and the braid 38.

The inner layer 36a is the same as the inner layer 6a of the first embodiment, and the outer layer 36b is also the same as the outer layer 6b of the first embodiment.

As illustrated in FIG. 9, the braid 38 is formed by alternately weaving a first wire wound in a first direction with respect to the inner layer 36a and a second wire wound in a second direction intersecting with the first direction into a mesh shape.

To be specific, the first wire of the present embodiment includes 12 first wires (39a, 39b, 39c, 39d, 39e, 39f, 39g, 39h, 39j, 39k, 39m, and 39n (hereinafter, referred to as “39a to 39n”)) wound in the first direction with respect to the inner layer 36a on the outer periphery of the inner layer 36a.

On the other hand, the second wire includes six third wires (37a, 37b, 37c, 37d, 37e, 37f (hereinafter, referred to as “37a to 37f”)) and 18 fourth wires consisting of six sets of three wires (35aa-35ca, 35ab-35cb, 35ac-35cc, 35ad-35cd, 35ae-35ce, 35af-35cf (hereinafter, referred to as “35aa-35cf”)), the third and fourth wires being wound in the second direction intersecting with the first direction on the outer periphery of the inner layer 36a.

Although the first wires 39a to 39e and 39m to 39n, the third wires 37a to 37c and 37f, and the fourth wires 35aa-35ca, 35ab-35cb, 35ac-35cc are not illustrated in FIG. 9, the first wires, the third wires, and the fourth wires of the present embodiment are arranged in the same manner as the first wires, the third wires, and the fourth wires in FIG. 2.

Herein, for example, one set of “35aa-35ca” means that three fourth wires of the wire 35aa, the wire 35ba, and the wire 35ca form one set and are twisted with respect to the first wire (for example, 39a to 39n).

Therefore, the above-described six sets of “35aa-35ca”, “35ab-35cb”, “35ac-35cc”, “35ad-35cd”, “35ae-35ce”, and “35af-35cf” mean that the respective three fourth wires form one set and are twisted with respect to the first wire (for example, 39a to 39n).

As illustrated in FIG. 9, each of the first wires 39a to 39n in the present embodiment is a wire having a substantially circular transverse section, each of the third wires 37a to 37f constituting the second wire is a flat wire (a wire rod having a substantially rectangular transverse section) with a width=W3 and a thickness=L1, and each of the fourth wires 35aa to 35cf constituting the second wire is a flat wire (a wire rod having a substantially rectangular transverse section) with a width=W4 smaller than the width W3 of the third wires 37a to 37f and a thickness=L1.

In the present embodiment, the width of each of the fourth wires 35aa to 35cf is set to be about ⅓ of the width of each of the third wires 37a to 37f.

When the catheter main body 33 constituting the catheter 30 according to the present embodiment is curved, each space between the fourth wires 35aa and 35ba, between the fourth wires 35ba and 35ca, between the fourth wires 35ab and 35bb, between the fourth wires 35bb and 35cb, between the fourth wires 35ac and 35bc, between the fourth wires 35bc and 35cc, between the fourth wires 35ad and 35bd, between the fourth wires 35bd and 35cd, between the fourth wires 35ae and 35be, between the fourth wires 35be and 35ce, between the fourth wires 35af and 35bf, and between the fourth wires 35bf and 35cf is slightly opened in a fan shape as illustrated in FIG. 7, whereby the flexibility and kink resistance of the catheter can be improved.

As materials of the twelve first wires 39a to 39n and the six third wires 37a to 37f and six sets of fourth wires 35aa to 35cf, which constitute the braid 38, biocompatible metallic wires such as stainless steel, tungsten, and Ni—Ti alloys can be used. In the present embodiment, stainless steel is used for both wires.

As illustrated in FIG. 9, the braid 38 used in the present embodiment has a form in which the first wires and the second wire are alternately woven into a mesh shape with one wire or one set each, but the present invention is not limited to such a braid, and like the catheter main body 13 constituting the catheter 10 illustrated in FIG. 5, it may be in a form in which the twelve first wires and the six third wires and six sets of fourth wires are alternately woven into a mesh shape with two wires each, or with one wire and one set each, and it can be also applied to a form in which the first wires and the second wires are alternately woven into a mesh shape with three or more wires each, two wires and one or more sets each, or one wire and two or more sets each.

In the present embodiment, explanation has been made assuming that the number of the first wires is 12, and the number of the second wires is six and six sets of three, but the present invention is not limited to these numbers, and the number of the first wires may be one or more, and the number of the second wires may be one and one set or more.

According to the catheter 30 of the present embodiment, there are included the inner layer 36a formed of the hollow tubular body having the lumen 3s, the braid 38 covering the inner layer 36a and constituted of the first wires 39a to 39n wound in the first direction and the second wires 37a to 37f and 35aa to 35cf wound in the direction intersecting the first direction, and the outer layer 36b covering the braid 38, and in particular, the second wires 37a to 37f and 35aa to 35cf are constituted by a combination of the third wires 37a to 37f having a width W3 of a predetermined length and the plurality of fourth wires 35aa to 35cf having a width W4 smaller than the width W3 of the third wires 37a to 37f, and therefore, the stiffness, flexibility, and kink resistance of the catheter 30 can be easily adjusted by adjusting the combination of the third wires 37a to 37f and the plurality of fourth wires 35aa to 35cf.

In addition, according to the catheter 30 of the present embodiment, since the third wires 37a to 37f and the plurality of fourth wires 35aa to 35cf are alternately arranged, it is possible to easily manufacture the catheter 30 in which the stiffness, flexibility, and kink resistance are appropriately adjusted.

In addition, according to the catheter 30 of the present embodiment, since each transverse section of the third wires 37a to 37f and the plurality of fourth wires 35aa to 35cf exhibits a substantially rectangular shape, a contact area with the inner layer 36a or the outer layer 36b can be increased without increasing an outer diameter of the catheter 30, and the kink resistance of the catheter 30 can be improved.

In addition, according to the catheter 30 of the present embodiment, since the transverse section of each of the first wires 39a to 39n exhibits a substantially circular shape, it is possible to increase the flexibility of the catheter 30 by reducing the contact areas between the first wires 39a to 39n and the third wires 37a to 37f or the plurality of fourth wires 35aa to 35cf.

### Fourth Embodiment

Next, a fourth embodiment of the present invention will be explained. Drawings to be used in the present embodiment are also exaggerated for ease of understanding, and dimensions thereof are different from the actual dimensions.

Hereinafter, the fourth embodiment of the present invention will be explained, but since an overall view of a catheter is the same as FIG. 1, the explanation thereof will be omitted, and portions common to the first embodiment will be denoted by the same reference numerals and the explanation thereof will be omitted.

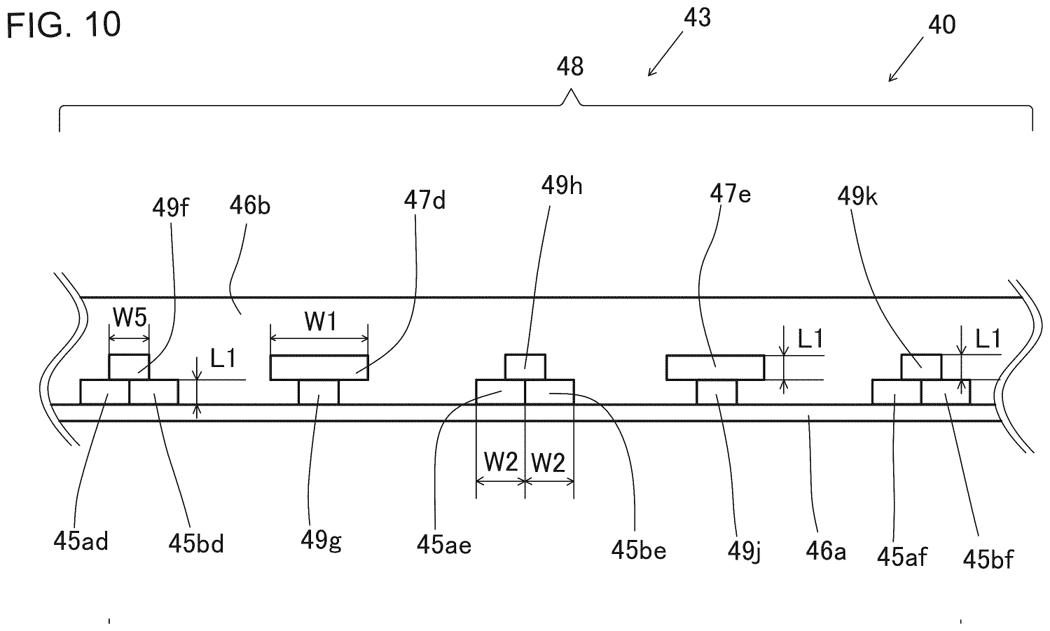

FIG. 10 is a view, corresponding to FIG. 6, of a braid constituting the catheter according to the fourth embodiment. A catheter 40 according to the fourth embodiment is different from the catheter 1 according to the first embodiment in the transverse sectional shape of the first wire. Namely, the transverse sectional shape of the first wire of the catheter 1 is substantially circular, whereas a transverse sectional shape of a first wire of the catheter 40 of the present embodiment is substantially rectangular.

The catheter 40 according to the present embodiment includes a catheter main body 43, a distal tip 2 joined to a distal end of the catheter main body 43, and a connector 4 joined to a proximal end of the catheter main body 43, similar to the features illustrated in FIG. 1.

As illustrated in FIG. 10, the catheter main body 43 includes an inner layer 46a having an inner cavity 3s, a braid 48 wound around an outer periphery of the inner layer 46a, and an outer layer 46b covering the inner layer 46a and the braid 48.

The inner layer 46a is the same as the inner layer 6a of the first embodiment, and the outer layer 46b is also the same as the outer layer 6b of the first embodiment.

As illustrated in FIG. 10, the braid 48 is formed by alternately weaving a first wire wound in a first direction with respect to the inner layer 46a and a second wire wound in a second direction intersecting with the first direction into a mesh shape.

To be specific, the first wire of the present embodiment includes 12 first wires (49a, 49b, 49c, 49d, 49e, 49f, 49g, 49h, 49j, 49k, 49m, 49n (hereinafter, referred to as “49a to 49n”)) wound in the first direction with respect to the inner layer 46a on the outer periphery of the inner layer 46a.

On the other hand, the second wire includes six third wires (47a, 47b, 47c, 47d, 47e, 47f (hereinafter, referred to as “47a to 47f”)) and 12 fourth wires consisting of six sets of wire pairs (45aa-45ba, 45ab-45bb, 45ac-45bc, 45ad-45bd, 45ae-45be, 45af-45bf (hereinafter, referred to as “45aa to 45bf”)), the third and fourth wires being wound in the second direction intersecting with the first direction on the outer periphery of the inner layer 46a.

Although the first wires 49a to 49e and 49m to 49n, the third wires 47a to 47c and 47f, and the fourth wires 45aa-45ba, 45ab-45bb, and 45ac-45bc are not illustrated in FIG. 10, the first wires, the third wires, and the fourth wires of the present embodiment are arranged in the same manner as the first wires, the third wires, and the fourth wires in FIG. 2.

Herein, for example, one set of “45aa-45ba” means that two fourth wires of the wire 45aa and the wire 45ba form one set and are twisted with respect to the first wire (for example, 49a to 49n).

Therefore, the above-described six sets of “45aa-45ba”, “45ab-45bb”, “45ac-45bc”, “45ad-45bd”, “45ae-45be”, and “45af-45bf” mean that the respective two fourth wires form one set and are twisted with respect to the first wire (for example, 49a to 49n).

As illustrated in FIG. 10, each of the first wires 49a to 49n in the present embodiment is a flat wire (a wire rod having a substantially rectangular transverse section) with a width=W5 and a thickness=L1, each of the third wires 47a to 47f constituting the second wire is a flat wire (a wire rod having a substantially rectangular transverse section) with a width=W1 and a thickness=L1, and each of the fourth wires 45aa to 45bf constituting the second wire is a flat wire (a wire rod having a substantially rectangular transverse section) with a width=W2 smaller than the width W1 of the third wires 47a to 47f and a thickness=L1.

In the present embodiment, the width of each of the fourth wires 45aa to 45bf is set to about ½ times the width of each of the third wires 47a to 47f.

When the catheter main body 43 constituting the catheter 40 of the present embodiment is curved, each space between the fourth wires 45aa and 45ba, between the fourth wires 45ab and 45bb, between the fourth wires 45ac and 45bc, between the fourth wires 45ad and 45bd, between the fourth wires 45ae and 45be, and between the fourth wires 45af and 45bf is slightly opened in a fan shape as illustrated in FIG. 7, whereby the flexibility and kink resistance of the catheter can be improved.

As materials of the twelve first wires 49a to 49n and the six third wires 47a to 47f and six sets of fourth wires 45aa to 45bf, which constitute the braid 48, biocompatible metallic wires such as stainless steel, tungsten, and Ni—Ti alloys can be used. In the present embodiment, stainless steel is used for both wires.

As illustrated in FIG. 10, the braid 48 used in the present embodiment has a form in which the first wires and the second wires are alternately woven into a mesh shape with one wire or one set each, but the present invention is not limited to such a braid, and like the catheter main body 13 constituting the catheter 10 illustrated in FIG. 5, it may be in a form in which the twelve first wires and the six third wires and six sets of fourth wires are alternately woven into a mesh shape with two wires each, or with one wire and one set each, and it can be also applied to a form in which the first wires and the second wires are alternately woven into a mesh shape with three or more wires each, two wires and one or more sets each, or one wire and two or more sets each.

In the present embodiment, explanation has been made assuming that the number of the first wires is 12, and the number of the second wires is six and six sets of two, but the present invention is not limited to these numbers, and the number of the first wires may be one or more, and the number of the second wires may be one and one set or more.

According to the catheter 40 of the present embodiment, there are included the inner layer 46a formed of the hollow tubular body having a lumen 3s, the braid 48 covering the inner layer 46a and constituted of the first wires 49a to 49n wound in the first direction and the second wires 47a to 47f and 45aa to 45bf wound in the direction intersecting the first direction, and the outer layer 46b covering the braid 48, and in particular, the second wires 47a to 47f and 45aa to 45bf are constituted by a combination of the third wires 47a to 47f having a width W1 of a predetermined length and the plurality of fourth wires 45aa to 45bf having a width W2 smaller than the width W1 of the third wires 47a to 47f, and therefore, stiffness, flexibility, and kink resistance of the catheter 40 can be easily adjusted by adjusting the combination of the third wires 47a to 47f and the plurality of fourth wires 45aa to 45bf.

In addition, according to the catheter 40 of the present embodiment, since the third wires 47a to 47f and the plurality of fourth wires 45aa to 45bf are alternately arranged, it is possible to easily manufacture the catheter 40 in which the stiffness, flexibility, and kink resistance are appropriately adjusted.

Furthermore, according to the catheter 40 of the present embodiment, each transverse section of the third wires 47a to 47f and the plurality of fourth wires 45aa to 45bf exhibits a substantially rectangular shape. Therefore, a contact area with the inner layer 46a or the outer layer 46b can be increased without increasing an outer diameter of the catheter 40, and the kink resistance of the catheter 40 can be improved.

### Fifth Embodiment

Next, a fifth embodiment of the present invention will be explained. Drawings to be used in the present embodiment are also exaggerated for ease of understanding, and dimensions thereof are different from the actual dimensions.

Hereinafter, the fifth embodiment of the present invention will be explained, but since an overall view of a catheter is the same as FIG. 1, explanation thereof will be omitted, and portions common to the first embodiment will be denoted by the same reference numerals, and explanation thereof will be omitted.

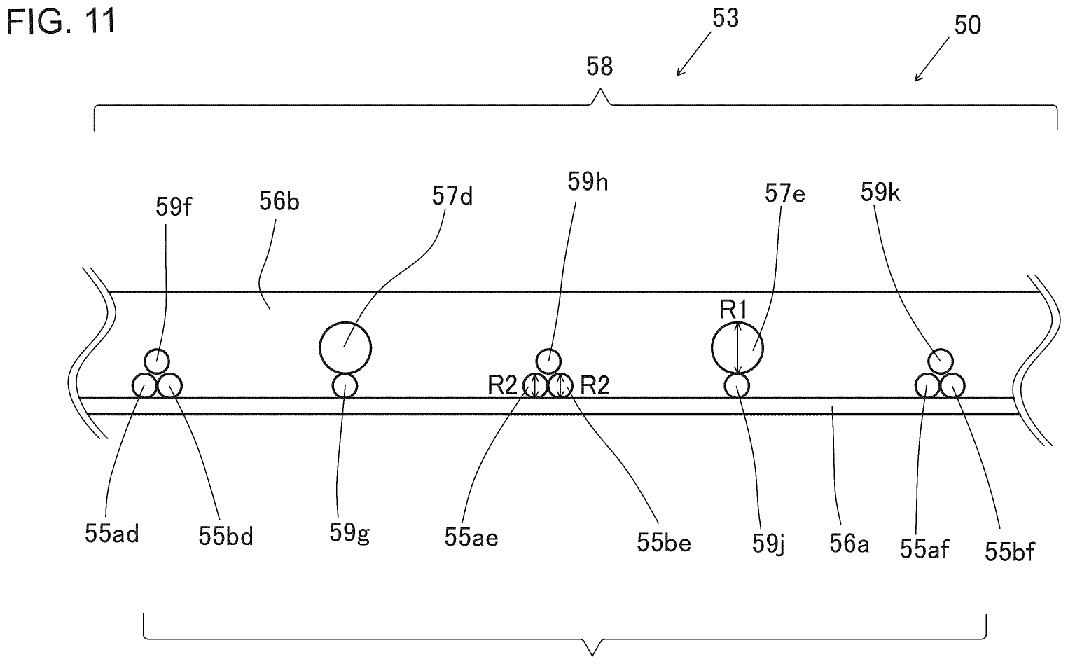

FIG. 11 is a view, corresponding to FIG. 6, of a braid constituting the catheter according to the fifth embodiment. A catheter 50 according to the fifth embodiment is different from the catheter 1 according to the first embodiment in the transverse sectional shape of the third wire and the fourth wire. Namely, the transverse sectional shape of the third wire and the fourth wire of the catheter 1 is substantially rectangular, whereas a transverse sectional shape of a third wire and a fourth wire of the catheter 50 of the present embodiment is substantially circular.

The catheter 50 according to the present embodiment includes a catheter main body 53, a distal tip 2 joined to a distal end of the catheter main body 53, and a connector 4 joined to a proximal end of the catheter main body 53, similar to the features shown in FIG. 1.

As illustrated in FIG. 11, the catheter main body 53 includes an inner layer 56a having an inner cavity 3s, a braid 58 wound around an outer periphery of the inner layer 56a, and an outer layer 56b covering the inner layer 56a and the braid 58.

The inner layer 56a is the same as the inner layer 6a of the first embodiment, and the outer layer 56b is also the same as the outer layer 6b of the first embodiment.

As illustrated in FIG. 11, the braid 58 is formed by alternately weaving a first wire wound in a first direction with respect to the inner layer 56a and a second wire wound in a second direction intersecting with the first direction into a mesh shape.

To be specific, the first wire of the present embodiment includes 12 first wires (59a, 59b, 59c, 59d, 59e, 59f, 59g, 59h, 59j, 59k, 59m, and 59n (hereinafter, referred to as “59a to 59n”)) wound in the first direction with respect to the inner layer 56a on the outer periphery of the inner layer 56a.

On the other hand, the second wire includes six third wires (57a, 57b, 57c, 57d, 57e, and 57f (hereinafter, referred to as “57a to 57f”)) and 12 fourth wires consisting of six sets of wire pairs (55aa-55ba, 55ab-55bb, 55ac-55bc, 55ad-55bd, 55ae-55be, and 55af-55bf (hereinafter, referred to as “55aa to 55bf”)), the third and fourth wires being wound in the second direction intersecting with the first direction on the outer periphery of the inner layer 56a.

Although the first wires 59a to 59e and 59m to 59n, the third wires 57a to 57c and 57f, and the fourth wires 55aa-55ba, 55ab-55bb, and 55ac-55bc are not illustrated in FIG. 11, the first wires, the third wires, and the fourth wires of the present embodiment are arranged in the same manner as the first wires, the third wires, and the fourth wires in FIG. 2.

Herein, for example, one set of “55aa-55ba” means that two fourth wires of the wire 55aa and the wire 55ba form one set and are twisted with respect to the first wire (for example, 59a to 59n).

Therefore, the above-described six sets of “55aa-55ba”, “55ab-55bb”, “55ac-55bc”, “55ad-55bd”, “55ae-55be”, and “55af-55bf” mean that the respective two fourth wires form one set and are twisted with respect to the first wire (for example, 59a to 59n).

As illustrated in FIG. 11, each of the first wires 59a to 59n in the present embodiment is a wire having a substantially circular transverse section, each of the third wires 57a to 57f constituting the second wire is a wire having a substantially circular transverse section with a diameter=R1, and each of the fourth wires 55aa to 55bf constituting the second wire is a wire having a substantially circular transverse section with a diameter=R2 smaller than the diameter=R1.

In the present embodiment, the diameter R2 of each of the fourth wires 55aa to 55bf is set to about ½ of the diameter R1 of each of the third wires 57a to 57f.

When the catheter main body 53 constituting the catheter 50 of the present embodiment is curved, each space between the fourth wires 55aa-55ba, between the fourth wires 55ab-55bb, between the fourth wires 55ac-55bc, between the fourth wires 55ad-55bd, between the fourth wires 55ae and 55be, and between the fourth wires 55af and 55bf is slightly opened as illustrated in FIG. 7, whereby the flexibility and kink resistance of the catheter can be improved.

As materials of the twelve first wires 59a to 59n and the six third wires 57a to 57f and six sets of fourth wires 55aa to 55bf, which constitute the braid 58, biocompatible metallic wires such as stainless steel, tungsten, and Ni—Ti alloys can be used. In the present embodiment, stainless steel is used for both wires.

As illustrated in FIG. 11, the braid 58 used in the present embodiment has a form in which the first wires and the second wires are alternately woven into a mesh shape with one wire or one set each, but the present invention is not limited to such a braid, and like the catheter main body 13 constituting the catheter 10 illustrated in FIG. 5, may be in a form in which the twelve first wires and the six third wires and six sets of fourth wires are alternately woven into a mesh shape with two wires each, or with one wire and one set each, and it can be also applied to a form in which the first wires and the second wires are alternately woven into a mesh shape with three or more wires each, two wires and one or more sets each, or one wire and two or more sets each.

In the present embodiment, explanation has been made assuming that the number of the first wires is 12, and the number of the second wires is six and six sets of two, but the present invention is not limited to these numbers, and the number of the first wires may be one or more, and the number of the second wires may be one and one set or more.

According to the catheter 50 of the present embodiment, there are included the inner layer 56a formed of the hollow tubular body having the lumen 3s, the braid 58 covering the inner layer 56a and constituted of the first wires 59a to 59n wound in the first direction and the second wires 57a to 57f and 55aa to 55bf wound in the direction intersecting the first direction, and the outer layer 56b covering the braid 58, and in particular, the second wires 57a to 57f and 55aa to 55bf are constituted by a combination of the third wires 57a to 57f having a transverse section diameter=R1 and the plurality of fourth wires 55aa to 55bf having a diameter R2 smaller than the diameter R1 of the third wires 57a to 57f, and therefore, stiffness, flexibility, and kink resistance of the catheter 50 can be easily adjusted by adjusting the combination of the third wires 57a to 57f and the plurality of fourth wires 55aa to 55bf.

In addition, according to the catheter 50 of the present embodiment, since the third wires 57a to 57f and the plurality of fourth wires 55aa to 55bf are alternately arranged, it is possible to easily manufacture the catheter 50 in which the stiffness, flexibility, and kink resistance are appropriately adjusted.

### Sixth Embodiment

Next, a sixth embodiment of the present invention will be explained. Drawings to be used in the present embodiment are also exaggerated for ease of understanding, and dimensions thereof are different from the actual dimensions.

Hereinafter, the sixth embodiment of the present invention will be explained, but since an overall view of a catheter is the same as FIG. 1, explanation thereof will be omitted, and portions common to the first embodiment will be denoted by the same reference numerals, and explanation thereof will be omitted.

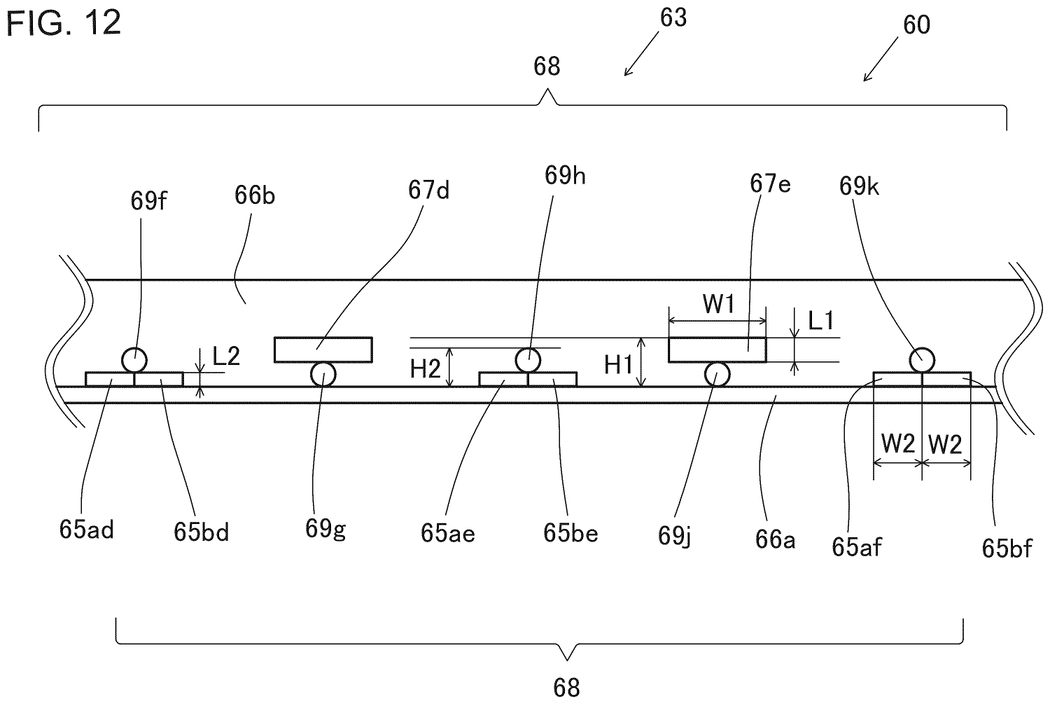

FIG. 12 is a view, corresponding to FIG. 6, of a braid constituting the catheter according to the sixth embodiment. A catheter 60 according to the sixth embodiment is different from the catheter 1 according to the first embodiment in the thickness of the fourth wire. Namely, the fourth wire of the catheter 1 is a flat wire (a wire rod having a substantially rectangular transverse section) with a width=W2 and a thickness=L1, whereas a fourth wire of the catheter 60 according to the present embodiment is a flat wire (a wire rod having a substantially rectangular transverse section) with a width=W2 and a thickness=L2 smaller than the thickness L1.

The catheter 60 according to the present embodiment includes a catheter main body 63, a distal tip 2 joined to a distal end of the catheter main body 63, and a connector 4 joined to a proximal end of the catheter main body 63, similar to the features shown in FIG. 1.

As illustrated in FIG. 12, the catheter main body 63 includes an inner layer 66a having an inner cavity 3s, a braid 68 wound around an outer periphery of the inner layer 66a, and an outer layer 66b covering the inner layer 66a and the braid 68.

The inner layer 66a is the same as the inner layer 6a of the first embodiment, and the outer layer 66b is also the same as the outer layer 6b of the first embodiment.

As illustrated in FIG. 12, the braid 68 is formed by alternately weaving a first wire wound in a first direction with respect to the inner layer 66a and a second wire wound in a second direction intersecting with the first direction into a mesh shape.

To be specific, the first wire of the present embodiment includes 12 first wires (69a, 69b, 69c, 69d, 69e, 69f, 69g, 69h, 69j, 69k, 69m, and 69n (hereinafter, referred to as “69a to 69n”) wound in the first direction with respect to the inner layer 66a on the outer periphery of the inner layer 66a.

On the other hand, the second wire includes six third wires (67a, 67b, 67c, 67d, 67e, 67f (hereinafter, referred to as “67a to 67f”) and twelve fourth wires consisting of six sets of wire pairs (65aa-65ba, 65ab-65bb, 65ac-65bc, 65ad-65bd, 65ae-65be, and 65af-65bf (hereinafter, referred to as “65aa to 65bf”)), the third and fourth wires being wound in the second direction intersecting with the first direction on the outer periphery of the inner layer 66a.

Although the first wires 69a to 69e and 69m to 69n, the third wires 67a to 67c and 67f, and the fourth wires 65aa-65ba, 65ab-65bb, and 65ac-65bc are not illustrated in FIG. 12, the first wires, the third wires, and the fourth wires of the present embodiment are arranged in the same manner as the first wires, the third wires, and the fourth wires in FIG. 2.

Herein, for example, one set of “65aa-65ba” means that two fourth wires of the wire 65aa and the wire 65ba form one set and are twisted with respect to the first wire (for example, 69a to 69n).

Therefore, the above-described six sets of “65aa-65ba”, “65ab-65bb”, “65ac-65bc”, “65ad-65bd”, “65ae-65be”, and “65af-65bf” mean that the respective two fourth wires form one set and are twisted with respect to the first wire (for example, 69a to 69n).

As illustrated in FIG. 12, each of the third wires 67a to 67f constituting the second wire in the present embodiment is a flat wire (a wire rod having a substantially rectangular transverse section) with a width=W1 and a thickness=L1, and each of the fourth wires 65aa to 65bf constituting the second wire is a flat wire (a wire rod having a substantially rectangular transverse section) with a width=W2 smaller than the width W1 of each of the third wires 67a to 67f and with a thickness=L2 smaller than the thickness L1 of each of the third wires 67a to 67f.

As a result, a height H2 from the inner layer 66a of a portion where the fourth wires 65aa to 65bf and the first wires 69a to 69n intersect is lower than a height H1 from the inner layer 66a of a portion where the third wires 67a to 67f and the first wires 69a to 69n intersect.

The outer layer 66b at the portion where the fourth wires 65aa to 65bf and the first wires 69a to 69n intersect is thicker than the outer layer 66b at the portion where the third wires 67a to 67f and the first wires 69a to 69n intersect, whereby flexibility of the catheter main body 63 constituting the catheter 60 can be further increased.

In the present embodiment, the width of each of the fourth wires 65aa to 65bf is set to about ½ times the width of each of the third wires 67a to 67f.

When the catheter main body 63 constituting the catheter 60 of the present embodiment is curved, each space between the fourth wires 65aa and 65ba, between the fourth wires 65ab and 65bb, between the fourth wires 65ac and 65bc, between the fourth wires 65ad and 65bd, between the fourth wires 65ae and 65be, and between the fourth wires 65af and 65bf is slightly opened in a fan shape as illustrated in FIG. 7, whereby the flexibility and kink resistance of the catheter can be improved.

As materials of the twelve first wires 69a to 69n and the six third wires 67a to 67f and six sets of fourth wires 65aa to 65bf, which constitute the braid 68, biocompatible metallic wires such as stainless steels, tungsten, and Ni—Ti alloys can be used. In the present embodiment, stainless steel is used for both wires.

As illustrated in FIG. 12, the braid 68 used in the present embodiment has a form in which the first wires and the second wires are woven into a mesh shape alternately with one wire or one set each, but the present invention is not limited to such a braid, and like the catheter main body 13 constituting the catheter 10 illustrated in FIG. 5, it may be in a form in which the twelve first wires and the six third wires and six sets of fourth wires are woven into a mesh shape alternately with two wires each, or one wire and one set each, and it is also applicable to a form in which the first wires and the second wires are alternately woven into a mesh shape with three or more wires each, two wires and one or more sets each, or one wire and two or more sets each.

In the present embodiment, explanation has been made assuming that the number of the first wires is 12, and the number of the second wires is six and six sets, but the present invention is not limited to these numbers, and the number of the first wires may be one or more, and the number of the second wires may be one and one set or more.

According to the catheter 60 of the present embodiment, there are included the inner layer 66a formed of a hollow tubular body having a lumen 3s, the braid 68 covering the inner layer 66a and constituted of the first wires 69a to 69n wound in the first direction and the second wires 67a to 67f and 65aa to 65bf wound in the direction intersecting the first direction, and the outer layer 66b covering the braid 68, and in particular, the second wires 67a to 67f and 65aa to 65bf are constituted by a combination of the third wires 67a to 67f having a width W1 of a predetermined length and the plurality of fourth wires 65aa to 65bf having a width W2 smaller than the width W1 of the third wires 67a to 67f, and therefore, stiffness, flexibility, and kink resistance of the catheter 60 can be easily adjusted by adjusting the combination of the third wires (67a to 67f) and the plurality of fourth wires (65aa to 65bf).

In addition, according to the catheter 60 of the present embodiment, since the third wires 67a to 67f and the plurality of fourth wires 65aa to 65bf are alternately arranged, it is possible to easily manufacture the catheter 60 in which the stiffness, flexibility, and kink resistance are appropriately adjusted.

In addition, according to the catheter 60 of the present embodiment, since each transverse section of the third wires 67a to 67f and the plurality of fourth wires 65aa to 65bf exhibits a substantially rectangular shape, a contact area with the inner layer 66a or the outer layer 66b can be increased without increasing an outer diameter of the catheter 60, whereby the kink resistance of the catheter 60 can be improved.

Further, according to the catheter 60 of the present embodiment, since a transverse section of each of the first wires 69a to 69n exhibits a substantially circular shape, contact areas between the first wires 69a to 69n and the third wires 67a to 67f or the plurality of fourth wires 65aa to 65bf is reduced, whereby the flexibility of the catheter 60 can be increased.

Furthermore, according to the catheter 60 of the present embodiment, since the height H2 from the inner layer 66a of the portion where the plurality of fourth wires 65aa to 65bf and the first wires 69a to 69n intersect is set to be lower than the height H1 from the inner layer 66a of the portion where the third wires 67a to 67f and the first wires 69a to 69n intersect, the flexibility of the catheter 60 can be further increased.

### Seventh Embodiment

Next, a seventh embodiment of the present invention will be explained. Drawings to be used in the present embodiment are also exaggerated for ease of understanding, and dimensions thereof are different from the actual dimensions.

Hereinafter, the seventh embodiment of the present invention will be explained, but since an overall view of a catheter is the same as FIG. 1, explanation thereof will be omitted, and portions common to the first embodiment will be denoted by the same reference numerals, and explanation thereof will be omitted.

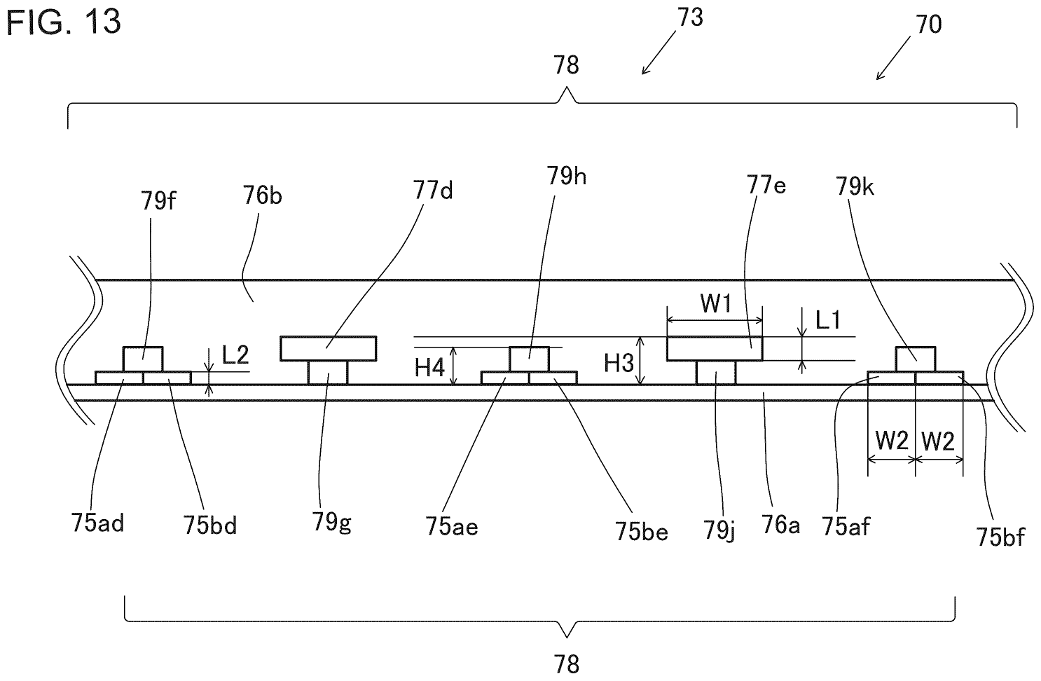

FIG. 13 is a view, corresponding to FIG. 6, of a braid constituting the catheter according to the seventh embodiment. A catheter 70 according to the seventh embodiment is different from the catheter 60 according to the sixth embodiment in the transverse sectional shape of the first wire. Namely, the transverse sectional shape of the first wire of the catheter 60 is substantially circular, whereas a transverse sectional shape of a first wire of the catheter 70 according to the present embodiment is substantially rectangular.

The catheter 70 according to the present embodiment includes a catheter main body 73, a distal tip 2 joined to a distal end of the catheter main body 73, and a connector 4 joined to a proximal end of the catheter main body 73, similar to the features shown in FIG. 1.

As illustrated in FIG. 13, the catheter main body 73 includes an inner layer 76a having an inner cavity 3s, a braid 78 wound around an outer periphery of the inner layer 76a, and an outer layer 76b covering the inner layer 76a and the braid 78.

The inner layer 76a is the same as the inner layer 6a of the first embodiment, and the outer layer 76b is also the same as the outer layer 6b of the first embodiment.

As illustrated in FIG. 13, the braid 78 is formed by alternately weaving a first wire wound in a first direction with respect to the inner layer 76a and a second wire wound in a second direction intersecting with the first direction into a mesh shape.

To be specific, the first wire of the present embodiment includes 12 first wires (79a, 79b, 79c, 79d, 79e, 79f, 79g, 79h, 79j, 79k, 79m, and 79n (hereinafter, referred to as “79a to 79n”) wound in the first direction with respect to the inner layer 76a on the outer periphery of the inner layer 76a.

On the other hand, the second wire includes six third wires (77a, 77b, 77c, 77d, 77e, and 77f (hereinafter, referred to as “77a to 77f”)) and twelve fourth wires consisting of six sets of wire pairs (75aa-75ba, 75ab-75bb, 75ac-75bc, 75ad-75bd, 75ae-75be, and 75af-75bf (hereinafter, referred to as “75aa to 75bf”)), the third and fourth wires being wound in the second direction intersecting with the first direction on the outer periphery of the inner layer 76a.

Although the first wires 79a to 79e and 79m to 79n, the third wires 77a to 77c and 77f, and the fourth wires 75aa-75ba, 75ab-75bb, and 75ac-75bc are not illustrated in FIG. 13, the first wires, the third wires, and the fourth wires of the present embodiment are arranged in the same manner as the first wires, the third wires, and the fourth wires in FIG. 2.

Herein, for example, one set of “75aa-75ba” means that two fourth wires of the wire 75aa and the wire 75ba form one set and are twisted with respect to the first wire (for example, 79a to 79n).

Therefore, the above-described six sets of “75aa-75ba”, “75ab-75bb”, “75ac-75bc”, “75ad-75bd”, “75ae-75be”, and “75af-75bf” mean that the respective two fourth wires form one set and are twisted with respect to the first wire (for example, 79a to 79n).

As illustrated in FIG. 13, each of the first wires 79a to 79n in the present embodiment is a flat wire (a wire rod having a substantially rectangular transverse section), each of the third wires 77a to 77f constituting the second wire in the present embodiment is a flat wire (a wire rod having a substantially rectangular transverse section) with a width=W1 and a thickness=L1, and each of the fourth wires 75aa to 75bf constituting the second wire is a flat wire (a wire rod having a substantially rectangular transverse section) with a width=W2 smaller than the width W1 of the third wires 77a to 77f and a thickness=L2 smaller than the thickness L1 of the third wires 77a to 77f.

As a result, a height H4 from the inner layer 76a of a portion where the fourth wires 75aa to 75bf and the first wires 79a to 79n intersect is lower than a height H3 from the inner layer 76a of a portion where the third wires 77a to 77f and the first wires 79a to 79n intersect.

The outer layer 76b at the portion where the fourth wires 75aa to 75bf and the first wires 79a to 79n intersect is thicker than the outer layer 76b at the portion where the third wires 77a to 77f and the first wires 79a to 79n intersect, whereby flexibility of the catheter main body 73 constituting the catheter 70 can be further increased.

In the present embodiment, the width of each of the fourth wires 75aa to 75bf is set to about ½ times the width of each of the third wires 77a to 77f.

When the catheter main body 73 constituting the catheter 70 of the present embodiment is curved, each space between the fourth wires 75aa and 75ba, between the fourth wires 75ab and 75bb, between the fourth wires 75ac and 75bc, between the fourth wires 75ad and 75bd, between the fourth wires 75ae and 75be, and between the fourth wires 75af and 75bf is slightly opened in a fan shape as illustrated in FIG. 7, whereby the flexibility and kink resistance of the catheter can be improved.

As materials of the twelve first wires (79a to 79n) and the six third wires (77a to 77f) and the six sets of fourthwires (75aa to 75bf), which constitute the braid 78, biocompatible metallic wires such as stainless steel, tungsten, and Ni—Ti alloys can be used. In the present embodiment, stainless steel is used for both wires.

As illustrated in FIG. 13, the braid 78 used in the present embodiment has a form in which the first wires and the second wires are alternately woven into a mesh shape with one wire or one set each, but the present invention is not limited to such a braid, and like the catheter main body 13 constituting the catheter 10 illustrated in FIG. 5, it may be in a form in which the twelve first wires and the six third wires and six sets of fourth wires are alternately woven into a mesh shape with two wires each, or one wire and one set each, and it can be also applied to a form in which the first wires and the second wires are alternately woven into a mesh shape with three or more wires each, two wires and one or more sets each, or one wire and two or more sets each.

In the present embodiment, explanation has been made assuming that the number of the first wires is 12, and the number of the second wires is six and six sets of two, but the present invention is not limited to these numbers, and the number of the first wires may be one or more, and the number of the second wires may be one and one set or more.

According to the catheter 70 of the present embodiment, there are included the inner layer 76a formed of a hollow tubular body having a lumen 3s, the braid 78 covering the inner layer 76a and constituted of the first wires 79a to 79n wound in the first direction and the second wires 77a to 77f and 75aa to 75bf wound in the direction intersecting the first direction, and the outer layer 76b covering the braid 78, and in particular, the second wires 77a to 77f and 75aa to 75bf are constituted by a combination of the third wires 77a to 77f having a width W1 of a predetermined length and the plurality of fourth wires 75aa to 75bf having a width W2 smaller than the width W1 of the third wires 77a to 77f, and therefore, stiffness, flexibility, and kink resistance of the catheter 70 can be easily adjusted by adjusting the combination of the third wires 77a to 77f and the plurality of fourth wires 75aa to 75bf.

In addition, according to the catheter 70 of the present embodiment, since the third wires 77a to 77f and the plurality of fourth wires 75aa to 75bf are alternately arranged, it is possible to easily manufacture the catheter 70 in which stiffness, flexibility, and kink resistance are appropriately adjusted.

Further, according to the catheter 70 of the present embodiment, since each transverse section of the third wires 77a to 77f and the plurality of fourth wires 75aa to 75bf exhibits a substantially rectangular shape, a contact area with the inner layer 76a or the outer layer 76b can be increased without increasing an outer diameter of the catheter 70, whereby the kink resistance of the catheter 70 can be improved.

Furthermore, according to the catheter 70 of the present embodiment, since the height H4 from the inner layer 76a of the portion where the plurality of fourth wires 75aa to 75bf and the first wires 79a to 79n intersect is set to be lower than the height H3 from the inner layer 76a of the portion where the third wires 77a to 77f and the first wires 79a to 79n intersect, the flexibility of the catheter 70 can be further increased.

### Eighth Embodiment

Next, an eighth embodiment of the present invention will be explained. Drawings to be used in the present embodiment are also exaggerated for ease of understanding, and dimensions thereof are different from the actual dimensions.

Hereinafter, the eighth embodiment of the present invention will be explained, but since an overall view of the catheter is the same as FIG. 1, explanation thereof will be omitted, and portions common to the first embodiment will be denoted by the same reference numerals, and explanation thereof will be omitted.

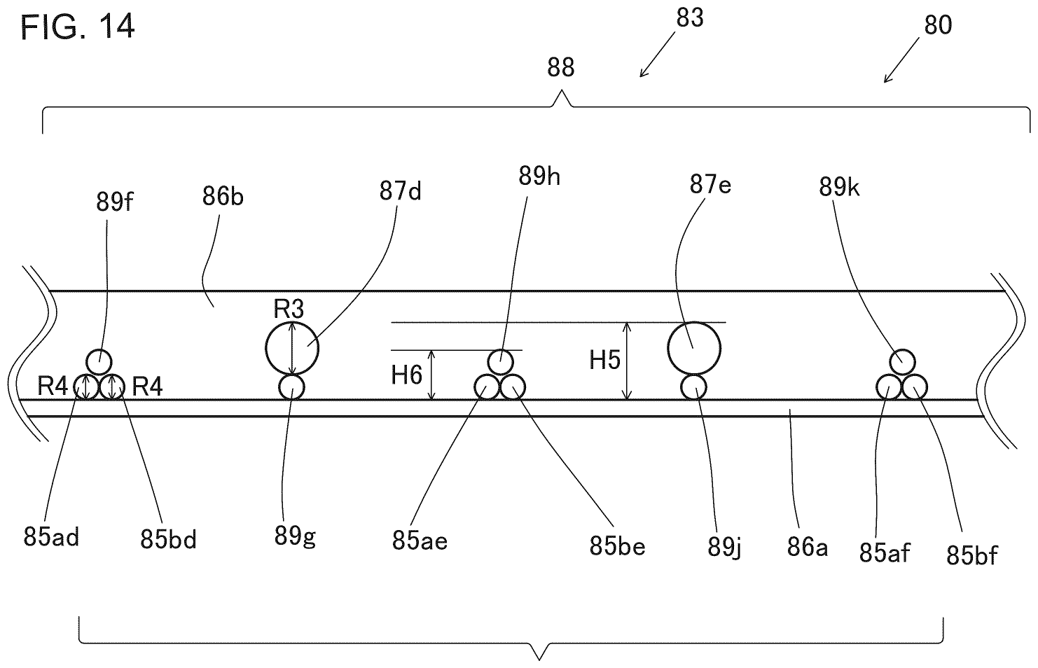

FIG. 14 is a view, corresponding to FIG. 6, of a braid constituting the catheter according to the eighth embodiment. A catheter 80 according to the eighth embodiment is different from the catheter 60 according to the sixth embodiment in the transverse sectional shape of the third wire and the fourth wire. Namely, the transverse sectional shape of the third wire and the fourth wire of the catheter 60 is substantially rectangular, whereas a transverse sectional shape of a third wire and a fourth wire of the catheter 80 according to the present embodiment is substantially circular.

The catheter 80 according to the present embodiment includes a catheter main body 83, a distal tip 2 joined to a distal end of the catheter main body 83, and a connector 4 joined to a proximal end of the catheter main body 83, similar to the features shown in FIG. 1.

As illustrated in FIG. 14, the catheter main body 83 includes an inner layer 86a having an inner cavity 3s, a braid 88 wound around an outer periphery of the inner layer 86a, and an outer layer 86b covering the inner layer 86a and the braid 88.

The inner layer 86a is the same as the inner layer 6a of the first embodiment, and the outer layer 86b is also the same as the outer layer 6b of the first embodiment.

As illustrated in FIG. 14, the braid 88 is formed by alternately weaving a first wire wound in a first direction with respect to the inner layer 86a and a second wire wound in a second direction intersecting with the first direction into a mesh shape.

To be specific, the first wire of the present embodiment includes 12 first wires (89a, 89b, 89c, 89d, 89e, 89f, 89g, 89h, 89j, 89k, 89m, and 89n (hereinafter, referred to as “89a to 89n”) wound in the first direction with respect to the inner layer 86a on the outer periphery of the inner layer 86a.

On the other hand, the second wire includes six third wires (87a, 87b, 87c, 87d, 87e, 87f (hereinafter, referred to as “87a to 87f”)) and twelve fourth wires consisting of six sets of wire pairs (85aa-85ba, 85ab-85bb, 85ac-85bc, 85ad-85bd, 85ae-85be, and 85af-85bf (hereinafter, referred to as “85aa to 85bf”)), the third and fourth wires being wound in the second direction intersecting with the first direction on the outer periphery of the inner layer 86a.

Although the first wires 89a to 89e and 89m to 89n, the third wires 87a to 87c and 87f, and the fourth wires 85aa-85ba, 85ab-85bb, and 85ac-85bc are not illustrated in FIG. 14, the first wires, the third wires and the fourth wires of the present embodiment are arranged in the same manner as the first wires, the third wires and the fourth wires in FIG. 2.

Herein, for example, one set of “85aa-85ba” means that two fourth wires of the wire 85aa and the wire 85ba form one set and are twisted with respect to the first wire (for example, 89a to 89n).

Therefore, the above-described six sets of “85aa-85ba”, “85ab-85bb”, “85ac-85bc”, “85ad-85bd”, “85ae-85be”, and “85af-85bf” mean that the respective two fourth wires form one set and are twisted with respect to the first wire (for example, 879a to 89n).

As illustrated in FIG. 14, each of the first wires 89a to 89n in the present embodiment is a wire having a substantially circular transverse section, each of the third wires 87a to 87f constituting the second wire is a wire having a substantially circular transverse section with a diameter=R3, and each of the fourth wires 85aa to 85bf constituting the second wire is a wire having a substantially circular transverse section with a diameter=R4 smaller than the diameter=R3.

As a result, a height H6 from the inner layer 86a of a portion where the fourth wires 85aa to 85bf and the first wires 89a to 89n intersect is lower than a height H5 from the inner layer 86a of a portion where the third wires 87a to 87f and the first wires 89a to 89n intersect.

The outer layer 86b at the portion where the fourth wires 85aa to 85bf and the first wires 89a to 89n intersect is thicker than the outer layer 86b at the portion where the third wires 87a to 87f and the first wires 89a to 89n intersect, whereby flexibility of the catheter main body 83 constituting the catheter 80 can be further increased.

In the present embodiment, the diameter R4 of each of the fourth wires 85aa to 85bf is set to about ½ of the diameter R3 of each of the third wires 87a to 87f.

When the catheter main body 83 constituting the catheter 80 of the present embodiment is curved, each space between the fourth wires 85aa and 85ba, between the fourth wires 85ab and 85bb, between the fourth wires 85ac and 85bc, between the fourth wires 85ad and 85bd, between the fourth wires 85ae and 85be, and between the fourth wires 85af and 85bf is slightly opened as illustrated in FIG. 7, whereby the flexibility and kink resistance of the catheter can be improved.

As materials of the twelve first wires 89a to 89n and the six third wires 87a to 87f and the six sets of fourth wires 85aa to 85bf, which constitute the braid 88, biocompatible metallic wires such as stainless steel, tungsten, and Ni—Ti alloys can be used. In the present embodiment, stainless steel is used for both wires.

As illustrated in FIG. 14, the braid 88 used in the present embodiment has a form in which the first wires and the second wires are alternately woven into a mesh shape with one wire or one set each, but the present invention is not limited to such a braid, and like the catheter main body 13 constituting the catheter 10 illustrated in FIG. 5, it may be in a form in which the twelve first wires and the six third wires and six sets of fourth wires are alternately woven into a mesh shape with two wires each, or one wire and one set each, and it can be also applied to a form in which the first wires and the second wires are alternately woven into a mesh shape with three or more wires each, two wires and one or more sets each, or one wire and two or more sets each.

In the present embodiment, the number of the first wires is 12, the number of the second wires is six and six sets of two. However, the present invention is not limited to these numbers, and the number of the first wires may be one or more, and the number of the second wires may be one or more and one set or more.

According to the catheter 80 of the present embodiment, there are included the inner layer 86a formed of the hollow tubular body having the lumen 3s, the braid 88 covering the inner layer 86a and constituted of the first wires 89a to 89n wound in the first direction and the second wires 87a to 87f and 85aa to 85bf wound in the direction intersecting the first direction, and the outer layer 86b covering the braid 88, and in particular, the second wires 87a to 87f and 85aa to 85bf are constituted by a combination of the third wires 87a to 87f having a transverse section diameter=R3 and the plurality of fourth wires 85aa to 85bf having a diameter R4 smaller than the diameter R3 of the third wires 87a to 87f, and therefore, stiffness, flexibility, and kink resistance of the catheter 80 can be easily adjusted by adjusting the combination of the third wires 87a to 87f and the plurality of fourth wires 85aa to 85bf.

In addition, according to the catheter 80 of the present embodiment, since the third wires 87a to 87f and the plurality of fourth wires 85aa to 85bf are alternately arranged, it is possible to easily manufacture the catheter 80 in which the stiffness, flexibility, and kink resistance are appropriately adjusted.

Furthermore, according to the catheter 80 of the present embodiment, since the height H6 from the inner layer 86a of the portion where the plurality of fourth wires 85aa to 85bf and the first wires 89a to 89n intersect is set to be lower than the height H5 from the inner layer 86a of the portion where the third wires 87a to 87f and the first wires 89a to 89n intersect, the flexibility of the catheter 80 can be further increased.

Although the catheters according to various embodiments of the present invention have been explained above, the present invention is not limited to the above-described embodiments, and various modifications can be made without departing from the scope of the present invention.

For example, in each of the above-described embodiments, explanation has been made assuming that two or three of the fourth wires form one set, but the number of the fourth wires forming one set may be four or more, and it is sufficient that a plurality of the fourth wires form one set.

However, in consideration of the manufacture of the catheter, the catheter can be most easily manufactured when two fourth wires form one set.

### Problem to be Solved

In the catheter described in Patent Literature 1, as illustrated in FIG. 3, since the wire rod 22a and the wire rod 22b different in stiffness are respectively arranged in a right-handed winding and a left-handed winding with respect to a longitudinal direction of the inner cylinder 21, there is a problem in that it is very difficult to arrange the wire rods at a time of manufacturing the braid.

It is also generally desirable to provide a catheter with improved flexibility and improved kink resistance.

One object of this disclosure is to provide a catheter in which stiffness, flexibility, and kink resistance of the catheter can be easily adjusted, the flexibility of the catheter is increased, and the kink resistance of the catheter is improved.

In order to address the above-described problem, a first aspect of the present invention is characterized in that a catheter includes an inner layer formed of a hollow tubular body, a braid covering the inner layer and constituted of a first wire wound in a first direction and a second wire wound in a direction intersecting the first direction, and an outer layer covering the braid, in which the second wire is constituted by a combination of a third wire having a width of a predetermined length and a plurality of fourth wires having a width smaller than the width of the third wire.

According to a second aspect of the present invention, in the catheter of the first aspect, the third wire and the plurality of fourth wires are alternately arranged.

According to a third aspect of the present invention, in the catheter according to the first aspect, each transverse section of the third wire and the fourth wire exhibits a substantially rectangular shape.

According to a fourth aspect of the present invention, in the catheter according to the second aspect, each transverse section of the third wire and the fourth wire exhibits a substantially rectangular shape.

According to a fifth aspect of the present invention, in the catheter according to the third aspect, a transverse section of the first wire exhibits a substantially circular shape.

According to a sixth aspect of the present invention, in the catheter according to the fourth aspect, a transverse section of the first wire exhibits a substantially circular shape.

According to a seventh aspect of the present invention, in the catheter according to any one of the first to sixth aspects, a height from the inner layer of a portion where the plurality of fourth wires and the first wire intersect is set to be lower than a height from the inner layer of a portion where the third wire and the first wire intersect.

According to the first aspect of the present invention, there is provided a catheter including an inner layer formed of a hollow tubular body, a braid covering the inner layer and constituted of a first wire wound in a first direction and a second wire wound in a direction intersecting the first direction, and an outer layer covering the braid, in which the second wire is constituted by a combination of a third wire having a width of a predetermined length and a plurality of fourth wires having a width smaller than the width of the third wire, and therefore, stiffness, flexibility, and kink resistance of the catheter can be easily adjusted by adjusting the combination of the third wires and the plurality of fourth wires.

According to the second aspect of the present invention, in the catheter of the first aspect, since the third wire and the plurality of fourth wires are alternately arranged, it is possible to easily manufacture a catheter in which stiffness, flexibility, and kink resistance are appropriately adjusted.

According to the third aspect of the present invention, in the catheter according to the first aspect, each transverse section of the third wire and the fourth wire exhibits a substantially rectangular shape, and therefore, in addition to an effect of the catheter of the first aspect, a contact area with the inner layer or the outer layer can be increased without increasing an outer diameter of the catheter, and the kink resistance of the catheter can be improved.

According to the fourth aspect of the present invention, in the catheter according to the second aspect, each transverse section of the third wire and the fourth wire exhibits a substantially rectangular shape, and therefore, in addition to an effect of the catheter of the second aspect, a contact area with the inner layer or the outer layer can be increased without increasing the outer diameter of the catheter, and the kink resistance of the catheter can be increased.

According to the fifth aspect of the present invention, in the catheter according to the third aspect, since the transverse section of the first wire exhibits a substantially circular shape, in addition to an effect of the catheter of the third aspect, the flexibility of the catheter can be increased by reducing a contact area between the first wire and the third wire or the plurality of fourth wires.

According to the sixth aspect of the present invention, in the catheter according to the fourth aspect, since the transverse section of the first wire exhibits a substantially circular shape, in addition to an effect of the catheter of the fourth aspect, the flexibility of the catheter can be increased by reducing a contact area between the first wire and the third wire or the plurality of fourth wires.

Furthermore, according to the seventh aspect of the present invention, in the catheter of any one of the first to sixth aspects, the height from the inner layer of the portion where the plurality of fourth wires and the first wire intersect is set to be lower than the height from the inner layer of the portion where the third wire and the first wire intersect, and therefore, the flexibility of the catheter can be further increased in addition to the effects of the catheter of any one of the first to sixth aspects.

### DESCRIPTION OF REFERENCE NUMERALS

1, 10, 20, 30, 40, 50, 60, 70, 80 Catheter
2 Distal tip
3, 13, 23, 33, 43, 53, 63, 73, 83 Catheter main body
4 Connector
5, 15, 25, 35, 45, 55, 65, 75, 85 Fourth wire
6a, 26a, 36a, 46a, 56a, 66a, 76a, 86a Inner layer
6b, 26b, 36b, 46b, 56b, 66b, 76b, 86b Outer layer
7, 17, 27, 37, 47, 57, 67, 77, 87 Third wire
8, 18, 28, 38, 48, 58, 68, 78, 88 Braid
9, 19, 29, 39, 49, 59, 69, 79, 89 First wire

1. A catheter comprising:
an inner layer formed of a hollow tubular body;
a braid covering the inner layer and including a first wire wound in a first direction and a second wire wound in a direction intersecting the first direction; and
an outer layer covering the braid, wherein
the second wire is constituted by a combination of a third wire having a width of a predetermined length and a plurality of fourth wires each having a width smaller than the width of the third wire.

2. The catheter according to claim 1, wherein the plurality of fourth wires are arranged as at least one set of grouped wires.

3. The catheter according to claim 1, wherein the plurality of fourth wires are arranged as a plurality of sets of grouped wires.

4. The catheter according to claim 1, wherein the plurality of fourth wires are arranged as a plurality of sets of two grouped wires.

5. The catheter according to claim 1, wherein the plurality of fourth wires are arranged as a plurality of sets of three grouped wires.

6. The catheter according to claim 1, wherein the plurality of fourth wires are arranged as a plurality of sets of grouped wires that are arranged to be adjacent and in contact with each other.

7. The catheter according to claim 1, wherein the third wire and the plurality of fourth wires are alternately arranged.

8. The catheter according to claim 1, wherein each transverse section of the third wire and the fourth wire has a substantially rectangular shape.

9. The catheter according to claim 7, wherein each transverse section of the third wire and the fourth wire has a substantially rectangular shape.

10. The catheter according to claim 8, wherein a transverse section of the first wire has a substantially circular shape.

11. The catheter according to claim 9, wherein a transverse section of the first wire has a substantially circular shape.

12. The catheter according to claim 1, wherein a height from the inner layer of a portion of the catheter where a plurality of the fourth wires and the first wire intersect is set to be lower than a height from the inner layer of a portion where the third wire and the first wire intersect.

13. The catheter according to claim 6, wherein a height from the inner layer of a portion of the catheter where a plurality of the fourth wires and the first wire intersect is set to be lower than a height from the inner layer of a portion where the third wire and the first wire intersect.

14. The catheter according to claim 7, wherein a height from the inner layer of a portion of the catheter where a plurality of the fourth wires and the first wire intersect is set to be lower than a height from the inner layer of a portion where the third wire and the first wire intersect.

15. The catheter according to claim 8, wherein a height from the inner layer of a portion of the catheter where a plurality of the fourth wires and the first wire intersect is set to be lower than a height from the inner layer of a portion where the third wire and the first wire intersect.
